(19) United States
(12) Patent Application Publication
(10) Pub. No.: US 2012/0029283 A1
(43) Pub. Date: Feb. 2, 2012

(54) GUIDE ASSEMBLY FOR ENDOSCOPE

(76) Inventors: Shinichi YAMAKAWA, Kanagawa (JP); Takayuki Nakamura, Kanagawa (JP); Masayuki Iwasaka, Kanagawa (JP)

(21) Appl. No.: 13/194,423

(22) Filed: Jul. 29, 2011

(30) Foreign Application Priority Data
Jul. 30, 2010 (JP) 2010-171920

(51) Int. Cl. A61B 1/01

(52) U.S. Cl. 600/114

(57) ABSTRACT

A guide assembly of a self-propelled type for an endoscope including an elongated tube for entry in a body cavity is provided. First and second self-propelled units are mounted on a steering device of the elongated tube in an axial direction thereof, for contacting a wall of the body cavity for propulsion. The first and second self-propelled units include respectively first and second driving devices, actuated with force by an external drive source, for converting the force for the propulsion. The first self-propelled unit includes a first torque coil structure for transmitting the force from the drive source to the first driving device. The second self-propelled unit includes a second torque coil structure for transmitting the force from the drive source to the second driving device discretely from the first torque coil structure. The first self-propelled unit is fixedly mounted on the steering device.

### BACKGROUND OF THE INVENTION

1. Field of the Invention

The present invention relates to a guide assembly for an endoscope. More particularly, the present invention relates to a guide assembly capable of causing an endoscope to enter a body cavity smoothly, and keeping a steering device in the endoscope steerable readily even with a self-propelled structure for guiding.

2. Description Related to the Prior Art

An endoscope is used to diagnose a body cavity, such as a large intestine in a gastrointestinal tract. Manipulation of the endoscope is a difficult process, because the large intestine is a tortuous organ in a human body, and some body parts are very changeable in the position in the body, such as a sigmoid colon and a transverse colon. Learning the manipulation of the endoscope of the large intestine requires much experience and time. If a doctor is insufficiently skilled in the manipulation, physical load to the body of a patient will be very large.

U.S. Pat. Nos. 6,971,990 and 7,736,300 (corresponding to JP-A 2009-513250) disclose a self-propelled apparatus for propelling the endoscope in the axial direction in the body cavity to facilitate the manipulation even for an unskilled operator or doctor. The self-propelled apparatus of the documents includes a movable endless track device or crawler device or toroidal device. The endless track device is driven to turn around for the endoscope to travel mechanically. Force of propulsion is created by the endless track device contacting a wall of the large intestine, so as to guide the endoscope deeply in the body cavity.

However, U.S. Pat. Nos. 6,971,990 and 7,736,300 disclose the self-propelled apparatus in which a support or housing of the endless track device longitudinally extends in the axial direction of the elongated tube. There is a problem in that the steering of the steering device is obstructed by the combined use of the guide assembly or the self-propelled apparatus, and that flexibility of the elongated tube may be lower. Accordingly, the manipulation may be more difficult.

### SUMMARY OF THE INVENTION

In view of the foregoing problems, an object of the present invention is to provide a guide assembly capable of causing an endoscope to enter a body cavity smoothly, and keeping a steering device in the endoscope steerable readily even with a self-propelled structure for guiding.

In order to achieve the above and other objects and advantages of this invention, a guide assembly of a self-propelled type for an endoscope including a section of an elongated tube for entry in a body cavity is provided. The elongated tube has a portion of a steering device on a distal side with a variable direction. At least first and second self-propelled units are mounted about the steering device, arranged with one another, for contacting a wall of the body cavity for propulsion. The first and second self-propelled units include respectively first and second driving devices, actuated with force by an external drive source, for converting the force into force for the propulsion.

The first self-propelled unit includes a first transmission device for transmitting the force from the drive source to the first driving device. The second self-propelled unit includes a second transmission device for transmitting the force from the drive source to the second driving device discretely from the first transmission device.

The first self-propelled unit is fixedly mounted on the steering device.

The first self-propelled unit is disposed on a distal side from the second self-propelled unit. Furthermore, a connecting coil structure connects the second self-propelled unit to the first self-propelled unit.

The connecting coil structure includes at least two coils having diameters different from one another, and combined by containing a first one in a second one thereof so that winding directions thereof are opposite to one another. Furthermore, a flexible tubular cover covers the connecting coil structure.

In one preferred embodiment, furthermore, the second self-propelled unit is fixedly mounted on the steering device.

Each of the at least first and second self-propelled units includes an endless track device, having an annular surface, driven by the first or second driving device with the force, for endlessly moving on an endless track in the axial direction.

Each of the at least first and second self-propelled units includes a worm gear sleeve, secured to the steering device, and rotated thereabout by the first or second transmission device. Each of the first and second driving devices includes an engagement roller, having teeth, rotatable about an axis perpendicular to the axial direction, meshed with the worm gear sleeve, for moving the endless track device.

The first self-propelled unit further includes a bearing sleeve for supporting the worm gear in a rotatable manner about the steering device. The worm gear of the second self-propelled unit is supported about the steering device in a rotatable manner with a small clearance.

In another preferred embodiment, each of the at least first and second self-propelled units further includes a bearing sleeve for supporting the worm gear in a rotatable manner about the steering device.

Each of the first and second driving devices includes a first ring sleeve disposed around the worm gear. A first through opening is formed in a wall of the first ring sleeve, for supporting the engagement roller in a rotatable manner. A second ring sleeve is disposed around the first ring sleeve, for movably supporting the endless track device. A second through opening is formed in a wall of the second ring sleeve. An idler roller is secured in the second through opening, for rotating about an axis perpendicular to the axial direction, and nipping the endless track device in cooperation with the engagement roller.

The idler roller is constituted by a pair of idler rollers, and the engagement roller is disposed between the idler rollers.

Each of the first and second transmission devices includes a torque coil structure having a proximal end connected to the drive source for transmitting torque. A pinion is secured to a distal end of the torque coil structure, for applying the torque to the first or second driving device when the drive source operates.

The endless track device is formed from fluid-impermeable material, and internally charged with liquid.

In one preferred embodiment, the endless track device is formed from fluid-impermeable material, and internally charged with gel.

In another preferred embodiment, the endless track device is formed from biocompatible plastic material.

The second self-propelled unit is operated remotely.

Consequently, it is possible to keep a steering device in the endoscope steerable readily even with a self-propelled structure for guiding, because of the combination of the two self-propelled units driven discretely.

### BRIEF DESCRIPTION OF THE DRAWINGS

The above objects and advantages of the present invention will become more apparent from the following detailed description when read in connection with the accompanying drawings, in which:

FIG. 1 is a plan illustrating an endoscope system;

FIG. 2 is a perspective view illustrating a guide assembly;

FIG. 3 is an exploded perspective view illustrating the guide assembly;

FIG. 4 is a vertical section illustrating the guide assembly;

FIG. 5 is a vertical section illustrating another preferred guide assembly without a connecting coil structure.

### DETAILED DESCRIPTION OF THE PREFERRED EMBODIMENT(S) OF THE PRESENT INVENTION

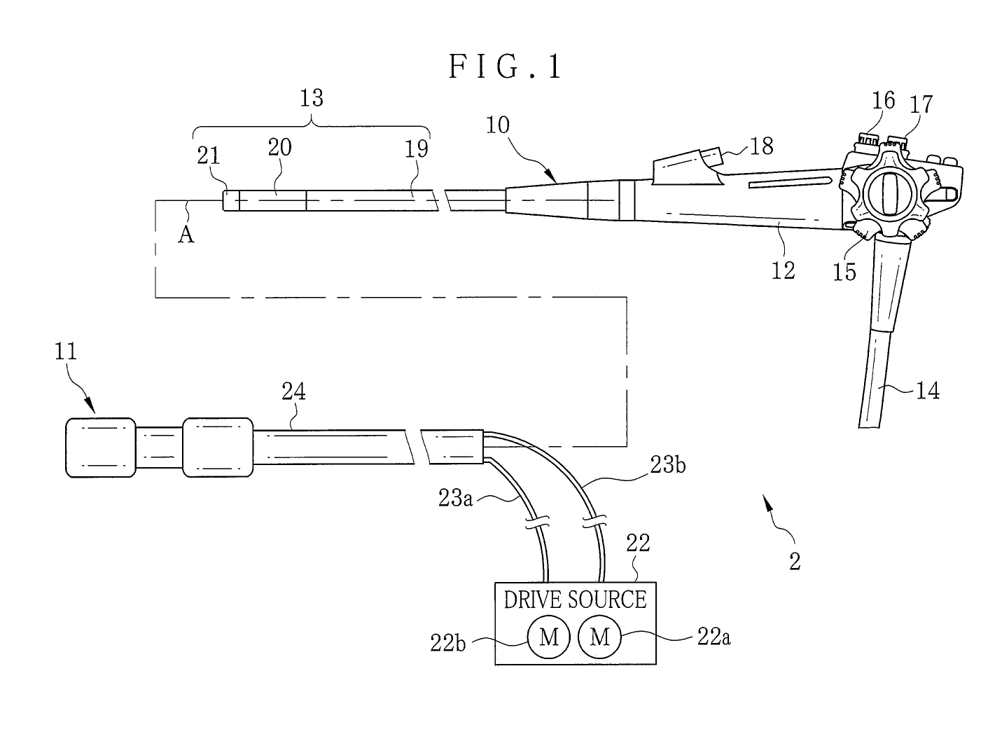

In FIG. 1, an endoscope system 2 includes an endoscope 10 and a self-propelled type of guide assembly 11. The endoscope 10 has a handle device 12 and an elongated tube 13 or guide tube disposed to extend from the handle device 12 for entry in a body cavity, for example, a large intestine of a gastrointestinal tract. A universal cable 14 is disposed to extend from the handle device 12. Connection plugs (not shown) are disposed at a proximal end of the universal cable 14 for connection with a light source apparatus and a processing apparatus (not shown) in a removal manner.

The handle device 12 includes steering wheels 15, an air/water button 16 and a suction button 17. The air/water button 16 is operable for supplying air or water through a distal end of the elongated tube 13. An instrument channel 18 is formed through the handle device 12 and the elongated tube 13 for receiving entry of a forceps, electrocautery device or other medical instrument.

The elongated tube 13 includes a flexible device 19, a steering device 20 and a head assembly 21 in a sequence in a distal direction from the handle device 12. The flexible device 19 has a length as great as several meters for reach of the head assembly 21 to an object of interest in a body cavity. The steering device 20 bends up and down and to the right and left in response to operation of the steering wheels 15 of the handle device 12. Thus, the head assembly 21 can be steered in a desired direction in the patient's body.

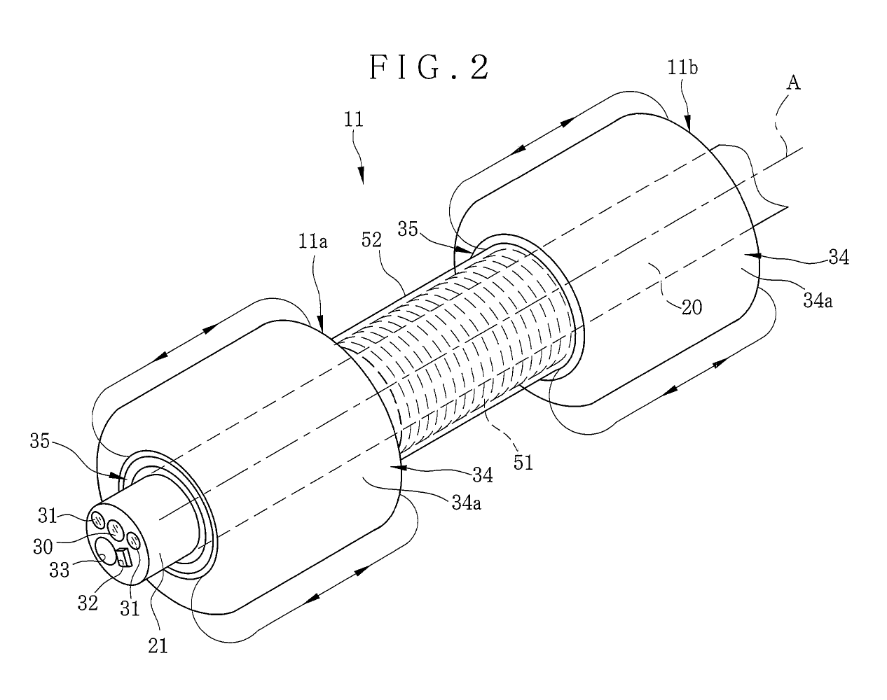

An imaging window 30 is formed in the head assembly 21 for imaging of a body part in the body. See FIG. 2. The head assembly 21 contains objective optics and an image sensor or solid-state image pickup device for imaging, such as CCD and CMOS image sensors. The image pickup device is connected to the processing apparatus by a signal line, which extends through the elongated tube 13, the handle device 12 and the universal cable 14. An object image of the body part is focused on a reception surface of the image pickup device, and is converted into an image signal. The processing apparatus processes the image signal from the image pickup device through the signal line by image processing, and obtains a video signal by conversion after the image processing. The object image is output and displayed on a monitor display panel (not shown) according to the video signal.

Various openings are formed in the head assembly 21 as illustrated in FIG. 2. Among those, a lighting window 31 passes illumination light from a light source apparatus toward an object of interest. An air/water nozzle 32 supplies air or water toward the imaging window 30 from an air/water supply device in the light source apparatus in response to depression of the air/water button 16. An instrument opening 33 causes a distal end of a medical instrument from the instrument channel 18 to appear distally.

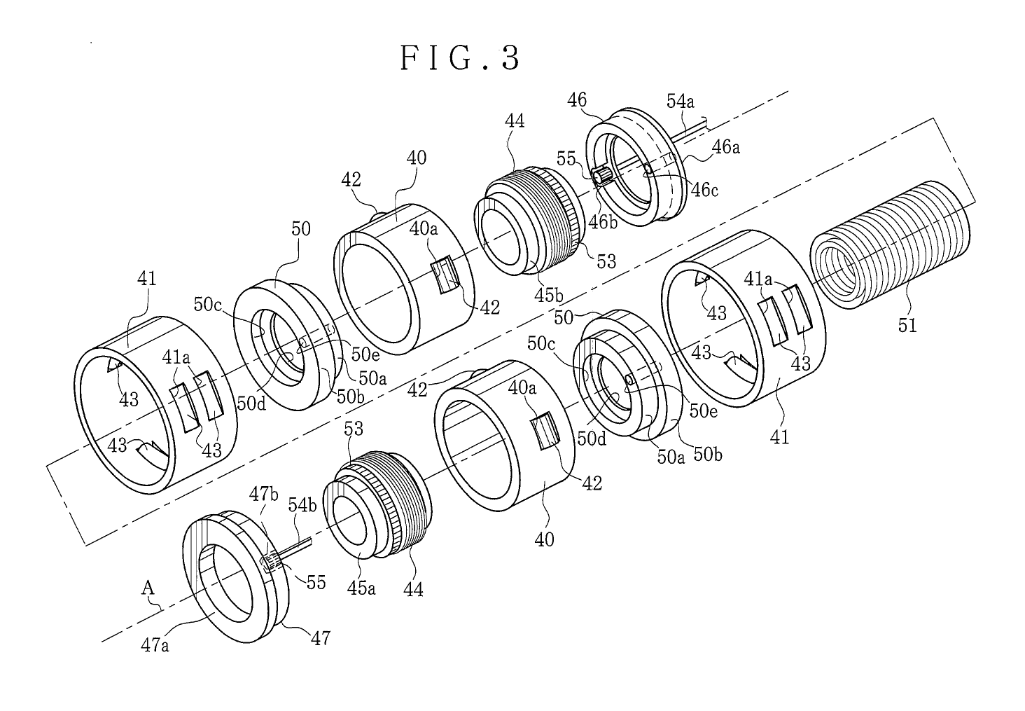

The guide assembly 11 is a device mounted on the endoscope 10, for assisting forward and backward movement of the elongated tube 13 of the endoscope 10 in a body cavity. There is an external drive source 22 which drives the guide assembly 11. The drive source 22 includes motors 22a and 22b. A torque coil structure 54a of a multi component type or three component type is connected with an output shaft of the motor 22a. A torque coil structure 54b of a multi component type or three component type is connected with an output shaft of the motor 22b. See FIG. 3. A protection sheath 23a receives entry of the torque coil structure 54a at its full length for covering. A protection sheath 23b receives entry of the torque coil structure 54b at its full length for covering. Torque of the motors 22a and 22b is transmitted to the torque coil structures 54a and 54b. Each of the torque coil structures 54a and 54b rotates inside the protection sheaths 23a and 23b responsively as transmission devices or wire devices.

An overtube 24 is used to cover the elongated tube 13, and is ready to expand and shrink in an axial direction of an axis A of the elongated tube 13. The protection sheaths 23a and 23b are entered between the overtube 24 and the elongated tube 13.

A controller (not shown) controls the drive source 22. A button panel (not shown) is connected to the controller. The button panel includes a command button for inputting command signals for forward movement, backward movement and stop of the self-propelled type of guide assembly 11, and a speed button for changing a moving speed of the guide assembly 11. Note that a control program can be prepared suitably for an object to be imaged. The drive source 22 can be actuated according to the control program without manipulating the button panel, so as to actuate the guide assembly 11 automatically.

In FIG. 2, the guide assembly 11 includes a first self-propelled unit 11a and a second self-propelled unit 11b or guide structures. The first self-propelled unit 11a is positioned on a distal side from the second self-propelled unit 11b. Those are secured to the steering device 20 of the elongated tube 13 of the endoscope 10. Each of the self-propelled units 11a and 11b includes a movable endless track device 34 or crawler device or toroidal device, and a driving device 35 or support device or barrel device with a rotating mechanism. The endless track device 34 has a hollow shape with an annular surface, is movable on an endless track, and is formed from a biocompatible plastic material having flexibility and fluid-impermeability. An example of the biocompatible plastic material is polyvinyl chloride. Also, polyamide resin, fluorocarbon resin, polyurethane resin and the like can be used. The inside of the endless track device 34 is filled with suitable fluid, such as liquid, gel, gas, or a combination of at least two of liquid, gel and gas.

The endless track device 34 of the first self-propelled unit 11a is driven by the motor 22a, and endlessly turns around in the axial direction of the axis A. The endless track device 34 of the second self-propelled unit 11b is driven by the motor 22b, and endlessly turns around in the axial direction. When an outer surface 34a of the endless track device 34 contacts an inner wall of a body cavity, propelling force occurs in the self-propelled units 11a and 11b to move the elongated tube 13 along the axis A.

To propel the elongated tube 13 in the distal direction, the elongated tube 13 is moved by the turn around of the endless track device 34 in an endless manner to orient the outer surface 34a in the proximal direction. To move the elongated tube 13 in the proximal direction, the endless track device 34 is turned around endlessly to orient the outer surface 34a in the distal direction.

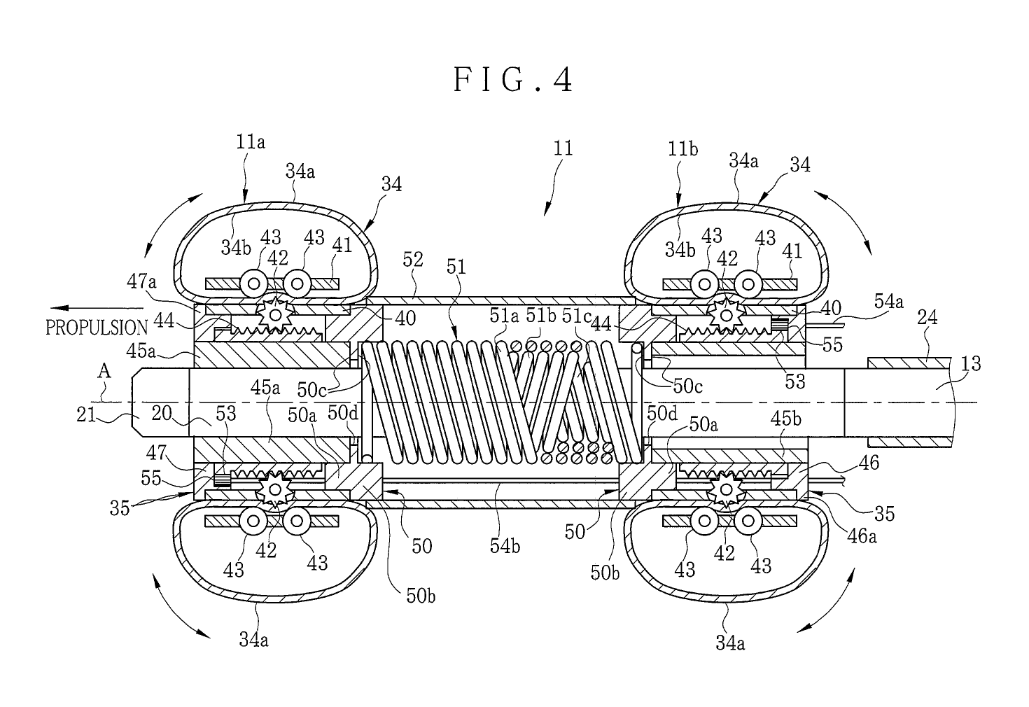

In FIGS. 3 and 4, the driving device 35 includes a first ring sleeve 40 and a second ring sleeve 41. Both of the first and second ring sleeves 40 and 41 are cylindrical, and have an equal size along the axis A. A diameter of the first ring sleeve 40 is smaller than that of the second ring sleeve 41. The first ring sleeve 40 is contained in and surrounded by the second ring sleeve 41. In FIG. 3, the endless track device 34 is not depicted.

Through openings 40a are formed in a wall of the first ring sleeve 40. An engagement roller 42 or drive roller or toothed roller has teeth, is disposed inside each of the through openings 40a, and rotates about an axis perpendicular to the axis A. The engagement roller 42 is disposed at the middle of the first ring sleeve 40 in the direction of the axis A. Three engagement rollers 42 are arranged at a pitch of 120 degrees in the circumferential direction of the first ring sleeve 40.

Through openings 41a are formed in a wall of the second ring sleeve 41. A pair of idler rollers 43 or driven rollers are disposed inside respectively the through openings 41a. Each of the idler rollers 43 is rotatable about an axis parallel to the axis of the engagement roller 42. Three pairs of idler rollers 43 are arranged at a pitch of 120 degrees in the circumferential direction of the second ring sleeve 41. When the first ring sleeve 40 is contained in the second ring sleeve 41, the second ring sleeve 41 is positioned relative to the first ring sleeve 40 to set the engagement roller 42 between the idler rollers 43. The endless track device 34 is mounted about the second ring sleeve 41 by passage in its end openings. The endless track device 34 is nipped between the engagement roller 42 and the idler rollers 43. An inner surface 34b of the endless track device 34 is contacted by the idler rollers 43, which are rotated by endless turn around of the endless track device 34.

Specifically, the endless track device 34 is prepared in the following manner. At first, a plastic tube having two open ends with flexibility and elasticity is initially formed from a sheet or film of the above-described suitable material. The plastic tube is halfway inserted in a sleeve lumen of the second ring sleeve 41. Then a portion of the plastic tube outside the sleeve lumen is bent back externally and extended to cover the periphery of the second ring sleeve 41. A first side line of the inserted half of the plastic tube is opposed to a second side line of the bent half to the plastic tube, so that the halves are attached together along the first and second side lines by adhesion, welding or other suitable method. Finally, the toroidal shape of the endless track device 34 is obtained.

A worm gear 44 or worm thread is contained in the first ring sleeve 40. The worm gear 44 is included in a worm gear sleeve (worm drive or worm sleeve). A bearing sleeve 45a or holding sleeve supports the worm gear 44 in the first self-propelled unit 11a. An inner sleeve 45b supports the worm gear 44 in the second self-propelled unit 11b. A bore of the inner sleeve 45b is larger than that of the bearing sleeve 45a. The elongated tube 13 of the endoscope 10 is entered in center holes of the bearing and inner sleeves 45a and 45b. The elongated tube 13 is tightly fitted in the bearing sleeve 45a. Thus, the bearing sleeve 45a is fixedly mounted on the steering device 20. The head assembly 21 projects distally from the bearing sleeve 45a upon mounting the bearing sleeve 45a on the elongated tube 13. The inner sleeve 45b is connected with the bearing sleeve 45a. Although the bearing sleeve 45a is immovable on the steering device 20, the inner sleeve 45b is rotatable on the steering device 20. Thus, the inner sleeve 45b is prevented from dropping away from the steering device 20. A worm thread of the worm gear 44 rotates about the bearing sleeve 45a or with the inner sleeve 45b along the axis A. The worm gear 44 is meshed with the engagement roller 42, which is rotated by the worm gear 44.

A rear end ring 46 is attached to the first ring sleeve 40 of the second self-propelled unit 11b. A flange 46a is a portion of the rear end ring 46 at its peripheral edge. The flange 46a, when the rear end ring 46 is attached to the first ring sleeve 40, contacts a rear edge of the first ring sleeve 40. The inner sleeve 45b is fitted in an inner hole of the rear end ring 46 in a tight manner without dropping.

A front end ring 47 is attached to the first ring sleeve 40 of the first self-propelled unit 11a. A flange 47a is a portion of the front end ring 47 at its peripheral edge. The flange 47a, when the front end ring 47 is attached to the first ring sleeve 40, contacts a front edge of the first ring sleeve 40. An end of the bearing sleeve 45a is fitted in an inner hole of the front end ring 47 in a tight manner without dropping.

A connecting ring 50 is fitted on the first ring sleeve 40 in the first self-propelled unit 11a by entry from a side opposite to the front end ring 47. Similarly, another connecting ring 50 is fitted on the first ring sleeve 40 in the second self-propelled unit 11b by entry from a side opposite to the rear end ring 46. The connecting ring 50 includes a small diameter portion 50a and a large diameter portion 50b, which are circular with different diameters. The small diameter portion 50a is firmly positioned in the first ring sleeve 40. The large diameter portion 50b is engaged with an edge of the first ring sleeve 40 upon fitting the small diameter portion 50a in the first ring sleeve 40. Recesses 50c are formed in the connecting ring 50 at front and rear ends. When the small diameter portion 50a is fitted in the first ring sleeve 40, each of the bearing and inner sleeves 45a and 45b is engaged with the inner surface of one of the recesses 50c. This prevents drop of the bearing and inner sleeves 45a and 45b. There is a connecting coil structure 51 of a multi component type or three component type, which has one end retained into a second one of the recesses 50c. A center opening 50d is formed in the connecting ring 50, and receives entry of the elongated tube 13.

The connecting coil structure 51 includes a first coil spring 51a, a second coil spring 51b and a third coil spring 51c. The first coil spring 51a is positioned externally. The second coil spring 51b has an outer diameter substantially equal to an inner diameter of the first coil spring 51a. The third coil spring 51c has an outer diameter substantially equal to an inner diameter of the second coil spring 51b. The coil springs 51a, 51b and 51c are combined in a multi layer form in such a state that their winding directions are different from one another. Specifically, the first and third coil springs 51a and 51c have turns wound in the counterclockwise direction. The second coil spring 51b has turns wound in the clockwise direction.

When the connecting coil structure 51 is rotated in the counterclockwise direction, the first and third coil springs 51a and 51c are shifted and tightened in an inward direction, the second coil spring 51b being shifted and loosened in an outward direction. When the connecting coil structure 51 is rotated in the clockwise direction, the first and third coil springs 51a and 51c are shifted and loosened in the outward direction, the second coil spring 51b being shifted and tightened in the inward direction. Thus, changes in relative positions of the first ring sleeve 40 in the first self-propelled unit 11a and the first ring sleeve 40 of the second self-propelled unit 11b can be prevented about the axis A even though the inner sleeve 45b in the second self-propelled unit 11b is not fixed to the steering device 20.

A tubular cover 52 is flexible along the axis A of the elongated tube 13, and has one end to which the connecting ring 50 is secured. The tubular cover 52 covers the connecting coil structure 51, and prevents body fluid from contacting the connecting coil structure 51. In FIG. 3, the tubular cover 52 is not depicted.

Spur gear teeth 53 or a driven gear is formed with a proximal end of the worm gear 44 in the second self-propelled unit 11b, the teeth being arranged about the axis A. A through hole (not shown) is formed in the rear end ring 46 in the direction of the axis A, and receives entry of the torque coil structure 54a. A pinion 55 is secured to an end of the torque coil structure 54a extending through the through hole. Thus, the pinion 55 is rotated together with the torque coil structure 54a. A cutout 46b is formed in the rear end ring 46, and contains the pinion 55. An axis of the pinion 55 is parallel to the axis A. The torque coil structure 54a includes three coil springs combined in a multi layer form in such a state that their winding directions are different from one another. The torque coil structure 54a can transmit torque even upon rotating in any of the forward and backward directions. The pinion 55 is meshed with the spur gear teeth 53. When the torque coil structure 54a rotates, the pinion 55 rotates responsively, to rotate the spur gear teeth 53.

In the first self-propelled unit 11a, the spur gear teeth 53 are formed with a distal end of the worm gear 44 and are arranged about the axis A. A receiving hole (not shown) is formed in the front end ring 47 in the direction of the axis A, and receives entry of the torque coil structure 54b. A pinion 55 is secured to an end of the torque coil structure 54b extending in the receiving hole. Thus, the pinion 55 is rotated together with the torque coil structure 54b. A cutout 47b is formed in the front end ring 47, and contains the pinion 55. An axis of the pinion 55 is parallel to the axis A. In a manner similar to the connecting coil structure 51, the torque coil structure 54b includes three coil springs combined in a multi layer form in such a state that their winding directions are different from one another. The torque coil structure 54b can transmit torque even upon rotating in any of the forward and backward directions. The pinion 55 is meshed with the spur gear teeth 53. When the torque coil structure 54b rotates, the pinion 55 rotates responsively, to rotate the spur gear teeth 53. A receiving hole 50e is formed in each connecting ring 50 and penetrates in the direction of the axis A. A receiving hole 46c is formed in the rear end ring 46 and penetrates in the direction of the axis A. The receiving holes 46c and 50e receive entry of the torque coil structure 54b. Thus, the torque coil structure 54b extends between the worm gear 44 and the first ring sleeve 40 in the second self-propelled unit 11b, between the connecting coil structure 51 and the tubular cover 52, and between the worm gear 44 and the first ring sleeve 40 in the first self-propelled unit 11a.

The operation of the endoscope system 2 is described now. At first, the overtube 24 is retained on the elongated tube 13 of the endoscope 10. The elongated tube 13 is entered in the bearing and inner sleeves 45a and 45b to mount the guide assembly 11 on the elongated tube 13.

After securing the overtube 24 and the guide assembly 11 to the endoscope 10, a power source of the processing apparatus, light source apparatus and controller is turned on. Then personal information of the patient is input. The elongated tube 13 of the endoscope 10 is entered in his of her body cavity.

After the head assembly 21 is advanced to a predetermined body part, for example, slightly short of a sigmoid colon, then the button panel is operated to turn on a power source for the drive source 22 of the self-propelled type of guide assembly 11 to drive the motors 22a and 22b. Then a command signal for start is input with the button panel. The motors 22a and 22b rotate the torque coil structures 54a and 54b in a predetermined direction. The pinion 55 is rotated by rotation of the torque coil structures 54a and 54b. The worm gear 44 in each of the self-propelled units 11a and 11b is rotated by the pinion 55 discretely.

When the worm gear 44 rotates together with the self-propelled units 11a and 11b, the engagement roller 42 is rotated responsively. Thus, the endless track device 34 endlessly turns around in each of the self-propelled units 11a and 11b. The guide assembly 11 advances in the axial direction of the elongated tube 13 when the outer surface 34a of the endless track device 34 contacts a wall of a body cavity. Consequently, the head assembly 21 advances along the wall of the body cavity.

When a command signal for a change is input by operating the button panel, the motors 22a and 22b change a rotational speed of the torque coil structures 54a and 54b. Thus, a moving speed of the self-propelled type of guide assembly 11 is changed. When a command signal for return is input by operating the button panel, the motors 22a and 22b cause the torque coil structures 54a and 54b to rotate in a backward direction, to move the guide assembly 11 and the head assembly 21 backwards. When a command signal for a stop is input by operating the button panel, the motors 22a and 22b stop to stop moving the guide assembly 11. It is possible to propel the head assembly 21 through the body cavity to an object of interest by suitably repeating those steps of the movement.

A doctor or operator steers the steering device 20 of the endoscope 10 by manipulating the steering wheels 15, to bend the head assembly 21 in a desired direction. Force for driving is applied to the second self-propelled unit 11b in the guide assembly 11 discretely from the force applied to the first self-propelled unit 11a. Thus, the self-propelled units 11a and 11b can follow the steering of the steering device 20. This is effective in keeping the steering device 20 free from being obstructed by the guide assembly 11. In the second self-propelled unit 11b, a clearance space exists between the inner surface of the inner sleeve 45b and the steering device 20, so as to facilitate smooth steering of the steering device 20. The second self-propelled unit 11b is connected to the first self-propelled unit 11a by the connecting coil structure 51. So the steering device 20 can operate for steering without being obstructed by the guide assembly 11.

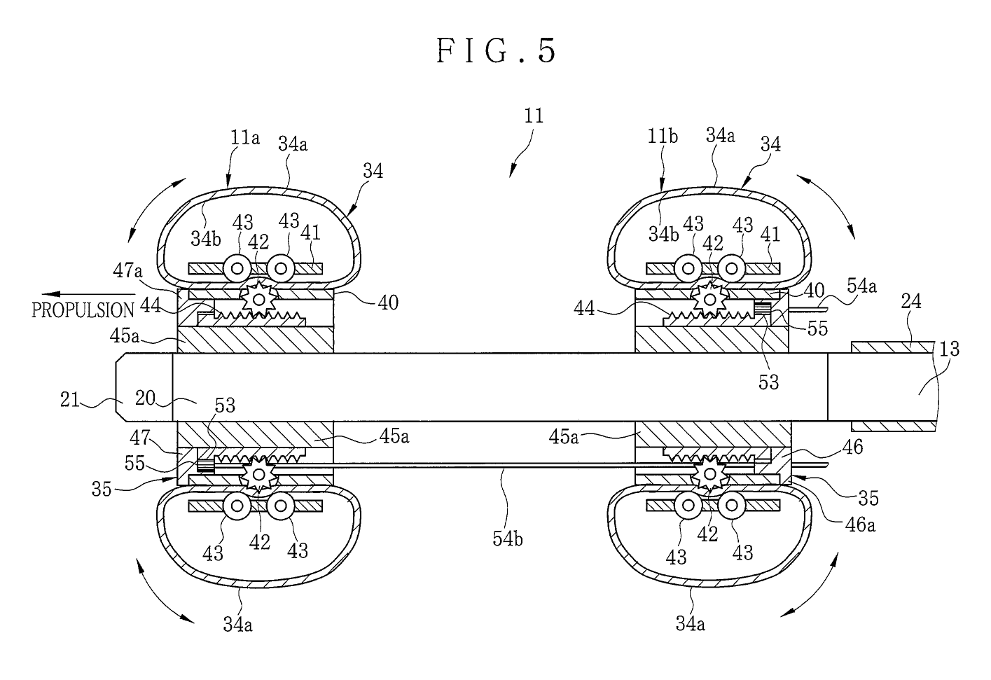

In the above embodiment, the first self-propelled unit 11a is fixedly mounted on the steering device 20. The second self-propelled unit 11b is connected to the first self-propelled unit 11a by the connecting coil structure 51. In contrast, another preferred embodiment having the self-propelled units 11a and 11b discretely mounted on the steering device 20 is illustrated in FIG. 5.

The connecting coil structure 51 is not present in the embodiment. To mount the self-propelled units 11a and 11b on the steering device 20, the bearing sleeve 45a in the second self-propelled unit 11b is mounted on the elongated tube 13. The bearing sleeve 45a is fixedly positioned on the steering device 20 without the play of the inner sleeve 45b above. The first self-propelled unit 11a is retained on the steering device 20 discretely from the second self-propelled unit 11b. Consequently, it is possible to reduce a manufacturing cost of the guide assembly 11 by eliminating the connecting ring 50 and the connecting coil structure 51.

In the embodiment, the connecting coil structure 51 is used between the two worm gears 44 in the self-propelled units 11a and 11b. However, other elements may be used between the two and having flexibility along the axis A, for example, only one coil spring, a rubber tube or the like.

Although the self-propelled units 11a and 11b are disclosed in the above embodiment, the number of the self-propelled units or guide structures may be three or more. Also, the number of the drive sources or motors for the self-propelled units may be one or three or more.

In the above embodiments, the self-propelled type of guide assembly is used with the endoscope for a medical use. Also, the guide assembly of the invention can be used with an endoscope for industrial use, an ultrasonic probe, or other instruments for imaging in a cavity. Although the movable endless track device or crawler device or toroidal device is turned around in the guide assembly, a guide assembly of the invention can be any mechanical type for entry in a body cavity as a component for an instrument for imaging.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A guide assembly of a self-propelled type for an endoscope including a section of an elongated tube for entry in a body cavity, said elongated tube having a portion of a steering device on a distal side with a variable direction, comprising:
at least first and second self-propelled units, mounted about said steering device, arranged with one another, for contacting a wall of said body cavity for propulsion;
wherein said first and second self-propelled units include respectively first and second driving devices, actuated with force by an external drive source, for converting said force into force for said propulsion.

2. A guide assembly as defined in claim 1, wherein said first self-propelled unit includes a first transmission device for transmitting said force from said drive source to said first driving device;
said second self-propelled unit includes a second transmission device for transmitting said force from said drive source to said second driving device discretely from said first transmission device.

3. A guide assembly as defined in claim 2, wherein said first self-propelled unit is fixedly mounted on said steering device.

4. A guide assembly as defined in claim 3, wherein said first self-propelled unit is disposed on a distal side from said second self-propelled unit;
further comprising a connecting coil structure for connecting said second self-propelled unit to said first self-propelled unit.

5. A guide assembly as defined in claim 4, wherein said connecting coil structure includes at least two coils having diameters different from one another, and combined by containing a first one in a second one thereof so that winding directions thereof are opposite to one another;
further comprising a flexible tubular cover for covering said connecting coil structure.

6. A guide assembly as defined in claim 3, wherein further said second self-propelled unit is fixedly mounted on said steering device.

7. A guide assembly as defined in claim 2, wherein each of said at least first and second self-propelled units includes an endless track device, having an annular surface, driven by said first or second driving device with said force, for endlessly moving in an axial direction of said elongated tube.

8. A guide assembly as defined in claim 7, wherein each of said at least first and second self-propelled units includes a worm gear sleeve, secured to said steering device, and rotated thereabout by said first or second transmission device;
each of said first and second driving devices includes an engagement roller, having teeth, rotatable about an axis perpendicular to said axial direction, meshed with said worm gear sleeve, for moving said endless track device.

9. A guide assembly as defined in claim 8, wherein said first self-propelled unit further includes a bearing sleeve for supporting said worm gear sleeve in a rotatable manner about said steering device;
said worm gear sleeve of said second self-propelled unit is supported about said steering device in a rotatable manner with a small clearance.

10. A guide assembly as defined in claim 8, wherein each of said at least first and second self-propelled units further includes a bearing sleeve for supporting said worm gear sleeve in a rotatable manner about said steering device.

11. A guide assembly as defined in claim 8, wherein each of said first and second driving devices includes:
a first ring sleeve disposed around said worm gear sleeve;
a first through opening, formed in a wall of said first ring sleeve, for supporting said engagement roller in a rotatable manner;
a second ring sleeve, disposed around said first ring sleeve, for movably supporting said endless track device;
a second through opening, formed in a wall of said second ring sleeve;
a first roller, secured in said second through opening, for rotating about an axis perpendicular to said axial direction, and nipping said endless track device in cooperation with said engagement roller.

12. A guide assembly as defined in claim 11, wherein said first roller is constituted by a pair of first rollers, and said engagement roller is disposed between said first rollers.

13. A guide assembly as defined in claim 2, wherein each of said first and second transmission devices includes:
a torque coil structure having a proximal end connected to said drive source for transmitting torque;
a pinion, secured to a distal end of said torque coil structure, for applying said torque to said first or second driving device when said drive source operates.

14. A guide assembly as defined in claim 2, wherein said endless track device is formed from fluid-impermeable material, and internally charged with liquid.

15. A guide assembly as defined in claim 2, wherein said endless track device is formed from fluid-impermeable material, and internally charged with gel.

16. A guide assembly as defined in claim 2, wherein said endless track device is formed from biocompatible plastic material.

17. A guide assembly as defined in claim 2, wherein said second self-propelled unit is operated remotely.
